(19) United States
(12) Patent Application Publication
(10) Pub. No.: US 2009/0280092 A1
(43) Pub. Date: Nov. 12, 2009

(54) G-Protein Coupled Receptor 83 As a Molecular Switch for the Induction of Regulatory (immunosuppressive) T-cells

(76) Inventors: Wiebke Hansen, Braunschweig (DE); Jan Buer, Cremlingen (DE)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/625,516

(22) Filed: Jan. 22, 2007

(51) Int. Cl. A61K 38/17; C12Q 1/04; C07K 14/435; A61K 31/7088; A61K 35/12; A61P 37/02; A61P 37/08

(52) U.S. Cl. 424/93.7; 435/34; 530/350; 514/12; 514/44 R

(57) ABSTRACT

The present invention makes use of the role of the G-protein coupled receptor 83 (GPCR83) in the induction of regulatory T cells (Tregs) during the course of ongoing immune response. The present invention relates to means and methods for identifying compounds that are interacting with the GPCR83 polypeptide, and to compounds capable of functioning as immunomodulators in mammals, in particular humans. In addition, the present invention relates to methods of treatment of a subject, in particular a human, suffering from an undesired immunoreaction.

The present invention makes use of the role of the G-protein coupled receptor 83 (GPCR83) in the induction of regulatory T cells (Tregs) during the course of ongoing immune response. The present invention relates to means and methods for identifying compounds that are interacting with the GPCR83 polypeptide, and to compounds capable of functioning as immunomodulators in mammals, in particular humans. In addition, the present invention relates to methods of treatment of a subject, in particular a human, suffering from an undesired immunoreaction.

### BACKGROUND OF THE INVENTION

G protein-coupled receptors (GPCRs), also known as seven transmembrane receptors, 7TM receptors, heptahelical receptors, or G protein linked receptors (GPLR), comprise a superfamily of membrane proteins in mammals that is characterized by a single polypeptide chain having seven transmembrane domains with an extracellular amino terminus and an intracellular carboxy terminus.

More than a thousand different GPCRs have been identified that respond to an enormous diversity of signaling molecules (ligands), including small peptides, lipid analogs, amino acid-derivatives and sensory stimuli, such as, for example, light, taste, and smell.

Despite the chemical and functional diversity of the signaling molecules that bind to GPCRs, each ligand produces a similar rearrangement of the amino acid regions that form the transmembrane core of the receptor. Portions of the cytoplasmic amino acid regions, together with the membrane-proximal region of the carboxy tail, mediate the binding to and the activation of the appropriate G protein, so-called because of its ability to bind guanine nucleotides.

G-proteins are trimers made up of the three subunits, alpha, beta, and gamma. Upon activation through its receptor, the alpha and beta-gamma subunits of the G-protein dissociate and bind to and modulate the activity of intracellular targets. Some G proteins subunits directly bind to ion channels, whereas others activate enzymes involved in a cytoplasmatic second messenger system. It is well established that such signal transduction pathways play important roles in many physiological and pathological processes.

For this reason, GPCRs are a very important class of drug targets that exist on the membrane surfaces of all cells. GPCRs are also associated with a wide range of therapeutic categories and diseases, including pain control and analgesia, asthma, inflammation, obesity, cancer, cardiovascular, metabolic, viral, immunomodulatory, gastrointestinal and central nervous system diseases. Although more than one thousand GPCRs with a potential therapeutic utility have been estimated in the human genome, to date there are only approximately two hundred well-characterized GPCRs with known ligands, of which only about half are currently targets of the development for commercial drugs. The remaining GPCRs, for which a ligand has not yet been identified, are typically referred to as “orphan GPCRs”.

Tregs, which are also known as suppressor T cells, are a specialized subpopulation of T cells, which act to suppress activation of the immune system and thereby maintain the immune system homeostasis and tolerance to self. In order to function properly, the immune system must discriminate between self and non-self. In case the self/non-self discrimination fails, the immune system will destroy cells and tissues of the body and, as a result, will cause autoimmune diseases. Tregs actively suppress such activation of the immune system and therefore prevent the pathological self-reactivity, i.e. the autoimmune disease. Therefore, Tregs play a critical role within the immune system and the immunosuppressive potential of these cells could be harnessed therapeutically in order to treat autoimmune diseases and facilitate transplantation tolerance, or to specifically eliminate cancer cells and/or to potentiate cancer immunotherapy.

Similar to other T cells, Tregs are developed in the thymus. In addition, Tregs can be also generated in the periphery, however the underlying molecular mechanism is not known yet. In order to define Tregs, the expression of the two CD4 and CD25 cell surface molecules is used, and these cells are often referred to as CD4⁺CD25⁺ Tregs. However, the use of CD25 as a marker for Tregs is problematic, since CD25 is also expressed on non-regulatory T cells in cases of immune activation, such as, for example, during an immune response to a pathogen. As identified through CD4 and CD25 expression, Tregs comprise about 5-10% of the mature CD4⁺ helper T cell subpopulation in mice and about 1-2% CD4⁺ helper T cells in humans.

Fontenot et al. (2005) have recently presented data arguing that the forkhead transcription factor Foxp3 acts as the Treg cell lineage specification factor and mediator of the genetic mechanism of dominant tolerance. In this study, it was shown that the expression of Foxp3 is highly restricted to the subset alpha-beta of T cells and, irrespective of CD25 expression, correlates with suppressor activity. In addition, it was shown that the induction of Foxp3 expression in non-regulatory T cells does not occur during pathogen-driven immune responses, and further that a Foxp3 deficiency does not impact the functional responses of non-regulatory T cells. Furthermore, it seems that T cell-specific ablation of Foxp3 is sufficient to induce the same early onset lymphoproliferative syndrome as observed in Foxp3-deficient mice. The analysis of Foxp3 expression during thymic development suggests that this mechanism is not hard-wired but is dependent on TCR/MHC ligand interactions. (Fontenot, J. D. et al., Immunity, 22(3):329-41 (2005)).

In addition, CD4⁺CD25⁺ regulatory T cells have also been referred to as “naturally-occurring” Tregs in order to distinguish them from “suppressor” T cell populations that are generated in vitro. In fact, the “naturally-occurring” CD4⁺CD25⁺ regulatory T cell population is a subset of the total Foxp3-expressing regulatory T cell population. The situation is further complicated by reports of additional “suppressor” T cell populations, including Tr1, CD8⁺ CD28⁻, and Qa-1 restricted T cells. However the contribution of these populations to self-tolerance and immune homeostasis is less well defined. Recent evidence suggests that mast cells may be important mediators of Treg-dependent peripheral tolerance.

In summary, it seems that expression of Foxp3 is required for Treg cell development, and appears to control a genetic program specifying this cellular fate. The large majority of Foxp3-expressing Tregs is found within the major histocompatibility complex (MHC) class II restricted CD4-expressing (CD4⁺) helper T cell population, and expresses high levels of the interleukin-2 receptor alpha chain (CD25). In addition to the Foxp3-expressing CD4⁺CD25⁺, there also appears to be a minor population of MHC class I restricted CD8⁺ Foxp3-expressing regulatory T cells.

Sugimoto et al. (2006) have shown that naturally occurring CD25(+)CD4(+) Tregs actively engage in the maintenance of immunologic self-tolerance and immunoregulation. They specifically express the transcription factor Foxp3 as a master control molecule for their development and function. Although several cell-surface molecules have been reported as Treg-specific markers, such as CD25, glucocorticoid-induced TNFR family-related gene/protein and CTL-associated molecule-4, they are also expressed on activated T cells derived from CD25(−)CD4(+) naive T cells. In order to identify Treg-specific molecules that are controlled by Foxp3, DNA microarray analysis was performed by comparing the following pairs of cell populations: fresh CD25(+)CD4(+) T cells versus fresh CD25(−)CD4(+) T cells, activated CD25(+)CD4(+) T cells versus activated CD25(−)CD4(+) T cells and retrovirally Foxp3-transduced CD25(−)CD4(+) T cells versus mock-transduced CD25(−)CD4(+) T cells.

It was found that the GPRC83, Ecm1, Cmtm7, Nkg7, Socs2 and glutaredoxin genes are predominantly transcribed in fresh and activated natural Treg as well as in Foxp3-transduced cells, while insulin-like 7, galectin-1, granzyme B and helios genes are natural Treg specific but Foxp3 independent. The GPRC83 expression on the cell surface of natural Tregs was confirmed by staining with a GPRC83-specific antibody. Retroviral transduction of either group of genes in CD25(−)CD4(+) T cells failed to confer in vitro suppressive activity. Thus, there are several genes that are expressed in a highly Treg-specific fashion. Some of these genes are controlled by Foxp3, and others are not. These genes, in particular, GPRC83, Ecm1 and Helios, could potentially be used as specific markers for natural Treg. (Sugimoto, N. et al., Int Immunol. 18(8):1197-209 (2006)).

An orphan GPCR of particular interest is the GPCR83. Although the amino acid sequence of this receptor has been previously disclosed (De Moerlooze L, et al. Cloning and chromosomal mapping of the mouse and human genes encoding the orphan glucocorticoid-induced receptor (GPR83). Cytogenet Cell Genet. 2000; 90(1-2):146-50; database Acc No: NP_{—}057624), neither its role in physiological and/or pathological processes has been elucidated, nor have the appropriate ligands for GPCR83 been identified. It has been recently shown that GPCR83 is up-regulated in regulatory Tregs. This gives a first hint that this receptor might be somehow involved in immune response(s). Thus, ligands of GPCR83 might be used therapeutically.

US 2006-0134109 very generally describes GPCR polypeptides and polynucleotides, recombinant materials, and transgenic mice, as well as methods for their production. The polypeptides and polynucleotides are described as useful, for example, in methods of diagnosis and treatment of diseases and disorders. The application also describes methods for identifying compounds (e.g., agonists or antagonists) using the GPCR polypeptides and polynucleotides, and for treating conditions associated with GPCR dysfunction with the GPCR polypeptides, polynucleotides, or identified compounds. The application also describes diagnostic assays for detecting diseases or disorders associated with inappropriate GPCR activity or levels.

As mentioned above, Tregs have an immunosuppressive potential which could be harnessed therapeutically. Therefore, the induction or expansion of Tregs for the treatment of autoimmune diseases or other undesired immunoreactions has been an aspect of immunological research in the last years.

It was shown that targeting of antigen specific T cells to immature dendritic cells in vivo leads to a relative increase of antigen-specific Foxp3+ regulatory T-cells that suppress the development of type 1 diabetes (Bruder, D. et al., Diabetes 54(12):3395-401 (2005)). Further, it was shown that prolonged subcutaneous infusion of low doses of antigen by means of osmotic pumps in a mouse transforms mature T cells into CD4+25+ Tregs that can persist for long periods of time in the absence of antigen and confer specific immunologic tolerance upon challenge with antigen (Apostolou, I. et al., J Exp Med. 199(10):1401-8 (2004)). It was also shown that different cytokines such as TGF-beta and IL-10 induce the development of T cells having an immunosuppressive potential (Chen, W. et al., J Exp Med. 198, 1875-1886 (2003); Fantini, M. C., J. Immunol. 172(9):5149-53 (2004)). In addition, the ectopic expression of the transcription factor Foxp3 results in the phenotypical modulation of conventional T cells. These T cells have both in vitro and in vivo regulatory potential and interfere with different diseases such as diabetes (Jeackel, E. et al., Diabetes, 54(2):306-10 (2005)) and contact allergy and systemic autoimmunity (Loser, K. et al., Gene Ther. 12(17):1294-304 (2005)).

US Patent application 2006-0002932 describes a method of enhancing an immune response in a subject, comprising administering to the subject a reagent that targets a cell having immunosuppressive activity, in an amount effective in reducing the immunosuppressive activity of the cell, thereby enhancing an immune response in the subject.

The methods for the induction of Tregs as described above require the knowledge of the antigen against which the immune response is directed. However, this knowledge is not available for most of the autoimmune diseases. In addition, the exact underlying molecular mechanism for the induction and expansion, respectively, of Tregs is not yet clarified. The in vitro induction of Tregs through treatment with different cytokines leads to the development of Tregs having a broad antigenic spectrum. In addition, in this approach the cells have to be removed from the subject and have to be cultivated and treated ex vivo, leading to problems which are due to the in vitro culture.

Thus, there is a need in the art to provide a target suitable to be used in order to identify compounds which could be effectively used for the induction of Tregs, whereby a knowledge regarding the specific antigen(s) would not be required.

In a recent study, the present inventors have shown that Foxp3 functions as a lineage specification factor for the development of naturally occurring thymus-derived CD4+CD25+ regulatory Tregs. Recent evidence suggests that naive Foxp3-CD4+CD25− T cells can be converted in the periphery into Foxp3+ Tregs. In this study, the inventors have identified the GPRC83 to be selectively up-regulated by CD4+CD25+ Tregs of both murine and human origin in contrast to naive CD4+CD25− or recently activated T cells. Furthermore, GPRC83 was induced upon overexpression of Foxp3 in naive CD4+CD25− T cells. Transduction of naive CD4+CD25− T cells with GPR83-encoding retroviruses did not confer in vitro suppressive activity. Nevertheless, GPR83-transduced T cells were able to inhibit the effector phase of a severe contact hypersensitivity reaction of the skin, indicating that GPRC83 itself or GPRC83-mediated signals conferred suppressive activity to conventional CD4+ T cells in vivo. Most strikingly, this in vivo acquisition of suppressive activity was associated with the induction of Foxp3 expression in GPRC83-transduced CD4+ T cells under inflammatory conditions. These results suggest that GPR83 might be critically involved in the peripheral generation of Foxp3+ Tregs in vivo (Hansen, W. et al., J Immunol, 177(1):209-15 (2006)).

Thus, the present inventors were able to show that GPCR83 plays a crucial role in the generation of Tregs. Further, the activation or inactivation of GPCR83 seems to be an essential step for the induction or suppression for the development of Tregs. Such induction was only observed during an immune response, therefore, using GPCR83 as a target an undesired generation of Tregs could be avoided.

In view of the above, it is an object of the present invention to provide means and methods for identifying compounds interacting with the GPCR83 polypeptide and for compounds capable to function as immunomodulators in mammals, and in particular in humans. In addition, it is an further object of the present invention to provide methods of treatment of a human suffering from an undesired immunoreaction.

### SUMMARY OF THE INVENTION

The object of the present invention, in one preferred embodiment thereof, is solved by a method for identifying a compound capable of interacting with a G-Protein coupled receptor 83 (GPCR83, GPR83, GPR72, or KIAA1540), comprising the steps of

a) contacting the GPCR83 polypeptide or a functional fragment thereof or a host cell recombinantly expressing the GPCR83 polypeptide or a functional fragment thereof with a candidate compound, and
b) determining whether said candidate compound interacts with said GPCR83 polypeptide.

The object of the present invention, in further preferred embodiment thereof, is solved by compound capable of interacting with the GPCR83 polypeptide, identified through a method according to the present invention.

In an additional embodiment of the present invention a method for identifying a compound capable to function as an immunomodulator is provided, comprising the steps of

a) contacting a host cell recombinantly expressing a GPCR83 polypeptide or a functional fragment thereof with a candidate compound that interacts with a GPCR83 polypeptide, in particular a candidate compound according to claim 5, and
b) detecting a response of said host cell compared to a control response as detected in the absence of said candidate compound,
wherein said response indicates that said candidate compound is capable of functioning as an immunomodulator.

In another embodiment thereof, the present invention provides an immunomodulator as identified through a method according to the present invention.

In a further preferred embodiment thereof, the present invention provides a method for identifying a compound capable to function as an immunomodulator, comprising the steps of

a) contacting conventional T-cells with a candidate compound interacting with a GPCR83 polypeptide, in particular with a candidate compound according to claim 5,
b) detecting the level of conversion of conventional T-cells into regulatory T-cells, and
c) comparing said level of conversion to a control level of conversion as detected in the absence of said candidate compound,
wherein the altered conversion into regulatory T-cells indicates that the candidate compound is capable of functioning as an immunomodulator.

In another preferred embodiment thereof, the present invention provides an immunomodulator as identified through a method according to the present invention.

In a further embodiment thereof, the present invention provides a pharmaceutical composition, comprising an effective amount of any of an immunomodulator according to the present invention as above, a GPCR83 polypeptide or a functional fragment thereof, a polynucleotide encoding a GPCR83 polypeptide or a functional fragment thereof, a vector containing a polynucleotide encoding a GPCR83 polypeptide or a functional fragment thereof, or a host cell recombinantly expressing a GPCR83 polypeptide or a functional fragment thereof, and a pharmaceutically acceptable carrier.

In a further preferred embodiment thereof, the present invention provides a method of treatment of a human suffering from an undesired immunoreaction, comprising administering to said human an effective amount of a pharmaceutical composition according to the present invention.

In a further embodiment the present invention concerns a method of treatment of a human suffering from an autoimmune disease, allergy and/or a transplant rejection, comprising the steps of

a) culturing peripheral blood cells of said human comprising conventional T-cells,
b) converting said conventional T-cells in vitro into regulatory T-cells by overexpression of a GPCR83 polypeptide in said conventional T-cells or by contacting said T-cells with an immunomodulator according to the present invention, and
c) re-introducing said converted regulatory T-cells into a human.

### DETAILED DESCRIPTION OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

As outlined above, the present invention is based on the findings about the role of GPCR83 for the induction of Tregs during the course of an ongoing immune response. The present inventors have shown that overexpression of GPCR83 in conventional T cells results in the conversion of these conventional T cells into Tregs during the course of an ongoing immune response in a mouse. Such conversion was not observed in healthy animals, why an undesired development of Tregs and thus an unspecific immunosuppression can be excluded.

The present inventors have also shown that surprisingly the overexpression of GPCR83 isoform 4, but not GPCR83 isoform 1, results in the induction of Tregs. Thus, the specific ligand of GPCR83, preferably of GPCR83 isoform 4 could be used therapeutically in order to treat autoimmune diseases, allergies and to facilitate transplantation tolerance.

Therefore, according to a first aspect of the present invention, provided is a method for identifying a compound capable of interacting with the with a G-Protein coupled receptor 83 (GPCR83), comprising the steps of a) contacting the GPCR83 polypeptide or a functional fragment thereof or a host cell recombinantly expressing the GPCR83 polypeptide or a functional fragment thereof with a candidate compound, and b) determining whether said candidate compound interacts with said GPCR83 polypeptide. Preferably, said method according to the present invention further comprises the step of c) selecting those candidate compounds that interact with said GPCR83 polypeptide or a functional fragment thereof.

In a preferred embodiment of the method according to present invention, said GPCR83 polypeptide is selected from the group of GPCR83 isoform 1 polypeptide, GPCR83 isoform 2 polypeptide, GPCR83 isoform 3 polypeptide, and GPCR83 isoform 4 polypeptide or a functional fragment thereof.

In the context of the present invention, “GPCR83 isoform peptides” shall mean the mammalian, preferably human, homologs of the mouse GPCR83 (Swiss-Prot entry P30731) as described in the databases and by Harrigan et al. (Harrigan M T, Campbell N F, Bourgeois S. Identification of a gene induced by glucocorticoids in murine T-cells: a potential G protein-coupled receptor. Mol. Endocrinol. 1991 September; 5(9):1331-8).

In a particularly preferred embodiment of the method according to present invention, the GPCR83 polypeptide is the GPCR83 isoform 4 polypeptide or a functional fragment thereof.

The term “functional fragment” of the GPCR83 polypeptide, in accordance with the present invention, shall mean a peptide, a protein, or a polypeptide which encompasses amino acid chains of a given length and which still exhibits essentially the same biological activity as the mature GPCR83 receptor. Preferably the polypeptide provides at least 20% (e.g., at least: 20%; 30%; 40%; 50%; 60%; 70%; 80%; 90%; 95%; 98%; 99%; 99.5%; or 100% or even more) of the biological activity of the full-length GPCR83 receptor. The same applies to the different isoforms of GPCR83, e.g. the term “GPCR83 isoform 4 polypeptide or a functional fragment thereof” in accordance with the present invention comprises a peptide, a protein, or a polypeptide which encompasses amino acid chains of a given length and which still exhibits essentially the same activity as the mature GPCR83 isoform 4 receptor. Preferably the polypeptide exhibits at least 20% (e.g., at least: 20%; 30%; 40%; 50%; 60%; 70%; 80%; 90%; 95%; 98%; 99%; 99.5%; or 100% or even more) of the activity of the full-length GPCR83 isoform 4 receptor. A fragment within the meaning of the present invention as above refers to one of the GPCR proteins bearing at least one N-terminal, C-terminal and/or internal deletion. The resulting fragment has a length of at least about 50, preferably of at least about 100, more preferably of at least about 150, more preferably of at least about 200, more preferably of at least about 250, more preferably of at least about 300, more preferably of at least about 350 and most preferably of at least about 400 amino acids.

The polypeptides useable in the method of the invention include all those as disclosed herein and functional fragments of these polypeptides. The terms “polypeptide” and “protein” are used interchangeably and mean any peptide-linked chain of amino acids, regardless of posttranslational modification. The polypeptides can also include fusion proteins that contain either a full-length GPCR83 polypeptide or a functional fragment of it, fused to an unrelated amino acid sequence. The unrelated sequences can add further functional domains or signal peptides. The same applies to the different isoforms of GPCR83.

The GPCR83 of the invention and its gene or cDNA can be used in screening assays for identification of compounds that modulate its activity and which may therefore be potential drugs. As above, useful proteins include wild-type and polymorphic GPCR83s or fragments thereof (e.g., an extracellular domain, an intracellular domain, or a transmembrane domain), in a recombinant form or endogenously expressed. Drug screens to identify compounds acting on a normally occurring or an exogenously expressed GPCR83 may employ any functional feature of the protein. In one example, the phosphorylation state or other post-translational modification is monitored as a measure of GPCR83 biological activity. In addition, drug screening assays may be based upon the ability of the protein to transduce a signal across a membrane or upon the ability to activate a G protein or another molecule. For example, the ability of a G protein to bind GTP may be assayed. Alternatively, a target of the G protein can be used as a measure of GPCR83 biological activity.

Methods for identifying compounds (e.g., agonists or antagonists) using the GPCR polypeptides, and for treating conditions associated with GPCR dysfunction with the GPCR polypeptides, polynucleotides, or identified compounds are extensively described and can be derived from US 2006-0134109, in particular in paragraphs [740] to [837] thereof, and are herewith incorporated by reference.

Drug screening assays can also be based upon the ability of the GPCR83 to interact with other proteins. Such interacting proteins can be identified by a variety of methods known in the art, including, for example, radioimmunoprecipitation, co-immunoprecipitation, co-purification, and yeast two-hybrid screening. Such interactions can be further assayed by means including but not limited to fluorescence polarization or scintillation proximity methods. Drug screens can also be based upon putative functions of a GPCR83 polypeptide deduced from structure determination (e.g., by x-ray crystallography) of the protein and comparison of its 3-D structure to that of proteins with known functions. Molecular modeling of compounds that bind to the protein using a 3-D structure may also be used to determine drug candidates. Drug screens can be based upon a function or feature apparent upon creation of a transgenic or knock-out mouse, or upon overexpression of the protein or protein fragment in mammalian cells in vitro. Moreover, expression of the GPCR83 in yeast or C. elegans allows for screening of candidate compounds in wild-type and polymorphic backgrounds, as well as screens for polymorphisms that enhance or suppress the GPCR83-dependent phenotype. Modifier screens can also be performed in a GPCR83 transgenic or knock-out mouse.

Assays of GPCR83 activity include binding to intracellular interacting proteins. Furthermore, assays may be based upon the molecular dynamics of macromolecules, metabolites, and ions by means of fluorescent-protein biosensors. Alternatively, the effect of candidate modulators on expression or activity may be measured at the level of GPCR83 production using the same general approach in combination with standard immunological detection techniques, such as western blotting or immunoprecipitation with a GPCR83 polypeptide-specific antibody. Again, useful modulators are identified as those that produce a change in GPCR83 polypeptide production. Modulators may also affect GPCR83 activity without any effect on expression level.

The test/candidate compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g, Houghten (1992) Biotechniques 13:412-421), oron beads (Lam (1991) Nature 354:82-84), chips (Fodor (1993) Nature 364:555-556), bacteria (Ladner U.S. Pat. No. 5,223,409), spores (Ladner U.S. Pat. No. 409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310).

Specific binding molecules, including natural ligands and synthetic compounds, can be identified or developed using isolated or recombinant GPCR83 products, GPCR83 variants, or preferably, cells expressing such products as above. Binding partners are useful for purifying GPCR83 products and detection or quantification of GPCR83 products in fluid and tissue samples using known immunological procedures. Binding molecules are also manifestly useful in modulating (i.e., blocking, inhibiting or stimulating) biological activities of a GPCR83 polypeptide, especially those activities involved in signal transduction. The DNA and amino acid sequence information provided by the present invention also makes possible identification of binding partner compounds with which a GPCR83 polypeptide or polynucleotide will interact. Methods to identify binding partner compounds include solution assays, in vitro assays wherein GPCR83 polypeptides are immobilized, and cell-based assays. Identification of binding partner compounds of GPCR83 polypeptides provides candidates for therapeutic or prophylactic intervention in pathologies associated with GPCR83 normal and aberrant biological activity.

As stated above, in a further aspect the present invention provides a method of isolating compounds interacting with a protein of the present invention comprising the steps of: a) contacting one or more of the GPCR83 proteins of the present invention, preferably one, with at least one potentially interacting compound, and b) measuring binding of said compound to said protein. This method is suitable for the determination of compounds that can interact with the proteins of the present invention and to identify, for example, inhibitors, activators, competitors or modulators of proteins of the present invention, in particular inhibitors, activators, competitors or modulators of the enzymatic activity of the proteins of the present invention.

The potentially interacting substance, whose binding to the protein of the present invention is to be measured, can be any chemical substance or any mixture thereof. For example, it can be a substance of a peptide library, a combinatory library, a cell extract, in particular a plant cell extract, a “small molecular drug”, a protein and/or a protein fragment as described herein.

The term “contacting” in the present invention means any interaction between the potentially binding substance(s) with the proteins of the invention, whereby any of the two components can be independently of each other in a liquid phase, for example in solution, or in suspension or can be bound to a solid phase, for example, in the form of an essentially planar surface or in the form of particles, pearls or the like. In a preferred embodiment a multitude of different potentially binding substances are immobilized on a solid surface like, for example, on a compound library chip and the protein of the present invention is subsequently contacted with such a chip. In another preferred embodiment the host cells recombinantly expressing the GPCR83 polypeptide or a functional fragment thereof, express the GPCR83 receptor at the cell surface and are contacted separately in small containers, e.g., microtitre plates, with various compounds. The same belongs to the different isoforms of GPCR83.

The proteins of the present invention employed in a method of the present invention can be a full length protein or a fragments thereof with N/C-terminal and/or internal deletions as described above.

Measuring of binding of the compound to the protein can be carried out either by measuring a marker that can be attached either to the protein or to the potentially interacting compound. Suitable markers are known to someone of skill in the art and comprise, for example, fluorescence or radioactive markers. The binding of the two components can, however, also be measured by the change of an electrochemical parameter of the binding compound or of the protein, e.g. a change of the redox properties of either the protein or the binding compound, upon binding. Suitable methods of detecting such changes comprise, for example, potentiometric methods. Further methods for detecting and/or measuring the binding of the two components to each other are known in the art, e.g. as described in US 2006-0134109, and can also be used to measure the binding of the potential interacting compound to the protein or protein fragments of the present invention. The effect of the binding of the compound or the activity of the protein can also be measured indirectly, for example, by assaying the phosphatase activity of the protein after binding.

As a further step after measuring the binding of a potentially interacting compound and after having measured at least two different potentially interacting compounds at least one compound can be selected, for example, on grounds of the measured binding activity or on grounds of the detected increase or decrease of protein activity, upon binding.

The thus selected binding compound is then, in a preferred embodiment, modified in a further step. Modification can be effected by a variety of methods known in the art, which include without limitation the introduction of novel side chains or the exchange of functional groups like, for example, introduction of halogens, in particular F, Cl or Br, the introduction of lower alkyl groups, preferably having one to five carbon atoms like, for example, methyl, ethyl, n-propyl, isopropyl, n-butyl, isobutyl, tert-butyl, n-pentyl or iso-pentyl groups, lower alkenyl groups, preferably having two to five carbon atoms, lower alkynyl groups, preferably having two to five carbon atoms or through the introduction of, for example, a group selected from the group consisting of NH₂, NO₂, OH, SH, NH, CN, aryl, heteroaryl, COH or COOH group.

The thus modified binding substances are than individually tested with the method of the present invention, i.e. they are contacted with the protein and subsequently binding of the modified compounds to the protein is measured. In this step both the binding per se can be measured and/or the effect of the function of the protein like. If needed the steps of selecting the binding compound, modifying the binding compound, contacting the binding compound with a protein of the invention and measuring the binding of the modified compounds to the protein can be repeated a third or any given number of times as required. The above described method is also termed “directed evolution” since it involves a multitude of steps including modification and selection, whereby binding compounds are selected in an “evolutionary” process optimizing its capabilities with respect to a particular property, e.g. its binding activity, its ability to activate, inhibit or modulate the activity of the GPCR83 according to the present invention.

The binding and/or interacting of candidate compounds may also be identified using yeast-two-hybrid systems.

The assays according to the present invention in general may be designed to screen large chemical libraries by automating the assay steps and providing compounds from any convenient source to assays, which are typically run in parallel (e.g., in microtiter formats on microtiter plates in robotic assays). The screening methods according to the present invention can be easily designed by the person skilled in the art on the basis of methods as described here, and the extensive literature in the field of screening (e.g. Szekeres P. G., Functional assays for identifying ligands at orphan G protein-coupled receptors. Receptor Channels. 2002; 8 (5-6): 297-308). For instance, the activity of the receptor described herein can be assessed using a variety of in vitro and in vivo assays to determine functional, chemical, and physical effects, e.g., measuring ligand binding, secondary messengers (e.g., cAMP, cGMP, IP₃, DAG, or Ca²⁺) ion flux, phosphorylation levels, transcription levels, of reporter constructs neurotransmitter levels, and the like.

Samples or assays that are treated with a potential receptor agonist may be compared to control samples without the test compound (agonist or antagonist), to examine the extent of modulation. Control samples (treated with agonists only) are assigned a relative receptor activity value of 100. Inhibition of receptor activity is achieved when the receptor activity value relative to the control is lower, and conversely receptor activity is enhanced when activity relative to the control is higher in the presence of identical amounts of the respective agonist.

The effects of the immunomodulator upon the function of the receptors can be measured by examining any of the parameters described above. Any suitable physiological change that affects receptor activity can be used to assess the influence of a test compound on the receptors of this invention. When the functional consequences are determined using intact cells or animals, one can measure a variety of effects such as changes in intracellular secondary messengers such as Ca²⁺, IP₃ or cAMP.

Preferred assays for G-protein coupled receptors include cells that are loaded with ion sensitive dyes to report receptor activity. In assays for identifying modulatory compounds, changes in the level of ions in the cytoplasm or membrane voltage will be monitored using an ion sensitive or membrane voltage fluorescent indicator, respectively. For G-protein coupled receptors, promiscuous G-proteins such as G_{α15} and G_{α16} and chimeric G-proteins can be used in the assay of choice (see, for example, Wilkie et al. (1991) Proc. Nat. Acad. Sci. USA 88: 10049-10053). Such promiscuous G-proteins allow coupling of a wide range of receptors to G-protein dependent signal pathways.

Receptor activation typically initiates subsequent intracellular events, e.g. increases in second messengers such as IP₃, which releases intracellular stores of calcium ions. Activation of some G-protein coupled receptors stimulates the formation of inositol trisphosphate through phospholipase C-mediated hydrolysis of phosphatidylinositol bisphosphate (Berridge & Irvine (1984) Nature 312: 315-21). IP₃ in turn stimulates the release of intracellular calcium ion stores. Thus, a change in cytoplasmic calcium ion levels, or a change in second messenger levels such as IP₃ can be used to assess G-protein coupled receptor function. Cells expressing such G-protein coupled receptors may exhibit increased cytoplasmic calcium levels as a result of contribution from both intracellular stores and via activation of ion channels, in which case it may be desirable, although not necessary, to conduct such assays in calcium-free buffer, optionally supplemented with a chelating agent such as EGTA, to distinguish fluorescence response resulting from calcium release from internal stores.

In a further aspect the present invention relates to a method for identifying a compound capable of functioning as an immunomodulator, comprising the steps of

a) contacting conventional T-cells with a candidate compound interacting with a GPCR83 polypeptide, in particular with a candidate compound as described herein,
b) detecting the level of conversion of conventional T-cells into regulatory T-cells, and
c) comparing said level of conversion to a control level of conversion as detected in the absence of said candidate compound,
wherein the altered conversion into regulatory T-cells indicates that the candidate compound is capable of functioning as an immunomodulator.

As used herein, “conventional T-cells” include cells defined by the presence of the cell surface marker CD4 and the absence of the surface marker CD25, as well as any other T-cells and/or cells that could be converted into Tregs.

In a preferred embodiment of the method according to present invention, the conventional T-cells naturally express a GPCR83 polypeptide, wherein said GPCR polypeptide is selected from the group of GPCR83 isoform 1 polypeptide, GPCR83 isoform 2 polypeptide, GPCR83 isoform 3 polypeptide, and GPCR83 isoform 4 polypeptide.

In a third aspect the present invention provides a method for identifying a compound capable of functioning as an immunomodulator, comprising the steps of

a) contacting a host cell recombinantly expressing a GPCR83 polypeptide or a functional fragment thereof with a candidate compound that interacts with a GPCR83 polypeptide, in particular a candidate compound as described herein, and
b) detecting a response of said host cell compared to a control response as detected in the absence of said candidate compound,
wherein said response indicates that said candidate compound is capable of functioning as an immunomodulator.

In one preferred embodiment of the method according to present invention, the GPCR83 polypeptide is selected from the group of GPCR83 isoform 1 polypeptide, GPCR83 isoform 2 polypeptide, GPCR83 isoform 3 polypeptide, and GPCR83 isoform 4 polypeptide or a functional fragment thereof.

In another preferred embodiment of the method according to present invention the GPCR83 polypeptide is the GPCR83 isoform 4 polypeptide or a functional fragment thereof.

As used herein, the term “response” shall mean the activation and/or inactivation of GPCR83. Such activation or inactivation of GPCR83 can be detected by measuring any changes of the biological activity of GPCR83. Methods for measuring the biological activity of GPCRs in vivo or in vitro are commonly known in the art and in addition, are described above and below. These methods can be applied to GPCR83 and comprise, for instance and without any limitation, the measurement of intracellular calcium level(s) or other parameters, such as IP3 or cAMP. Furthermore, also electrophysiological methods and transcription assays known in the art that are also suitable in order to measure the biological activity of GPCR83.

In a particularly preferred embodiment of the method according to the present invention, the candidate compound is selected from the group of neuropeptides. Neuropeptides are a therapeutically important class of GPCR ligands which represent signaling molecules in the nervous system of most organisms, including mammals. A neuropeptide according to the present invention may be derived from a family selected from the group of opioid, neurohypophyseal, tachykinins, bombesin/gastrin releasing peptide (GRP), secretins, insulins, somatostatins, gastrins, neuropeptide y, and derivates thereof. Further, the neuropeptide according to the present invention is derived from a precursor selected from the group comprising pro-opiomelanocortin (POMC), pro-.enkephalin, prodynorphin, provasopressin, pro-oxytocin, alpha-protachykinin a, beta-protachykinin a, gamma-protachykinin a, protachykinin b, probombesin, pro GPR, proglucagon, pro vasoactive intestinal peptide (VIP), pro growth hormone-releasing factor (GRF), pro-insulin, prosomatostatin, progastrin, procholecystokinin, pro neuropeptide y (NPY), pro pancreatic polypeptide (PP), pro peptide yy (PYY), Pro corticotrophin-releasing factor (CRF), procalcitonin, pro calcitonin gene-related peptide (CGRP), pro angiotensin, probradykinin, pro thyrotropin-releasing hormone (TRH), and derivates thereof. In addition, the neuropeptide according to the present invention is selected from the group comprising corticotrophin (ACTH), beta-lipotropin, alpha-MSH, alpha-endorphin, beta-endorphin, gamma-endorphin, met-enkephalin, leu-enkephalin, alpha-neoendorphin, beta-neoendorphin, dynorphin a. dynorphin b (rimorphin), leumorphin, vasopressin, neurophysin I, neurophyin II, oxytocin, substance p, neurokinin a, neuropeptide k, neuropeptide gamma, neurokinin b, bombesin, GRP, secretin, motilin, glucagons, VIP, GRF, insulin, insulin-like growth factors, somatostatin, gastrin, cholecystokinin (CCK), NPY, PP, PYY, CRF, calcitonin, CGRP, angiotensin, bradykinin, TRH, neurotensin, galanin, luteinizing hormone-releasing hormone (LHRH), and derivates thereof. Further preferred candidate compounds can be selected from the group of mast cell products, such as prostaglandins. Prostaglandins are well known in the literature, the predominant naturally occurring prostaglandins all have two double bonds and are synthesised from arachidonic acid (5, 8, 11, 14 eicosatetraenoic acid). The 1 series and 3 series are produced by the same pathway with fatty acids having one fewer double bond (8, 11, 14 eicosatrienoic acid or one more double bond (5, 8, 11, 14, 17 eicosapentaenoic acid) than arachidonic acid. Further preferred candidate compounds can be selected from the group of glucocorticoids. Glucocorticoids are also well known in the literature as a group of hormones including a series of synthetic products—prednisone, prednisolone, methylprednisolone, and dexamethasone—used, for example, in the treatment of some lymphocytic leukemias, lymphomas, and myeloma. Natural glucocorticoids are produced by the adrenal glands.

In another important aspect thereof, the present invention provides a compound capable of interacting with the GPCR83 polypeptide, identified through a method according to the present invention as above. The compound identified according to the present invention can serve as a lead compound in order to further develop compounds that are capable of functioning as immunomodulators, or can directly be used as a compound capable of functioning as an immunomodulator.

As used herein, the term “immunomodulator” comprises a substance, a compound or a composition which is of chemical or biological origin, and which has an influence on the induction or conversion of Tregs. Such influence on the induction or conversion of Tregs is based on the ability of the immunomodulator, to bind and/or to interact with the GPCR83 according to the present invention. The binding and/or interacting of the immunomodulator with the GPCR83 results in a change of the biological activity of GPCR83, leading to the induction or suppression of an immunoreaction. Thus, an immunomodulator according to the present invention comprises inducers or suppressors of an immunoreaction. An immunomodulator which functions as an inducer of an immunoreaction activates GPCR83, which finally results in the induction of Tregs during an ongoing immunoreaction. An immunomodulator which functions as a suppressor of an immunoreaction blocks the activity of GPCR83, which finally results in little or no induction/production of Tregs during an ongoing immunoreaction and thus leads to a decrease of an undesired suppression of an immunoreaction.

An immunomodulator according to the present invention occurs either naturally and/or is synthetically, recombinantly and/or chemically produced. Thus, an immunomodulator may be a protein, a protein-fragment, a peptide, an amino acid and/or derivatives thereof or other compound, such as ions.

An “immunomodulator” according to the present invention is a substance, a compound or a composition which is of chemical or biological origin, and which naturally occurs and/or which is synthetically, recombinantly and/or chemically produced. Thus, an immunomodulator may be a protein, a protein-fragment, a peptide, an amino acid and/or derivatives thereof or other compounds, such as ions, which bind to and/or interact with the mature GPCR83 as identified according to the present invention.

In a preferred embodiment of the method according to present invention, an immunomodulator comprises compounds selected from inducers or suppressors of an immunoreaction. An immunomodulator which functions as an inducer of an immunoreaction activates the GPCR83 receptor which finally results in an induction of Tregs during an ongoing immunoreaction. An immunomodulator which functions as a suppressor of an immunoreaction blocks the GPCR receptor activity leading to a decrease of an undesired suppression of an immunoreaction, since Tregs will not be produced.

In another aspect thereof, the present invention provides a host cell that recombinantly expresses the GPCR83 polypeptide or an isoform or a functional fragment thereof. The host cells that may be used for purposes of the invention include, but are not limited to, prokaryotic cells, such as bacteria (for example, E. coli and B. subtilis), which can be transformed with, for example, recombinant bacteriophage DNA, plasmid DNA, or cosmid DNA expression vectors containing the polynucleotide molecules encoding the GPCR83 polypeptide or said isoform or a functional fragment thereof; eukaryotic cells like yeast (for example, Saccharomyces and Pichia), which can be transformed with, for example, recombinant yeast expression vectors containing the nucleic acid molecule encoding the GPCR83 polypeptide or isoform or a functional fragment thereof, insect cell systems like, for example, Sf9 of Hi5 cells, which can be infected with, for example, recombinant virus expression vectors (for example, baculovirus) containing the nucleic acid molecules encoding the GPCR83 polypeptide or isoform or a functional fragment thereof; Xenopus oocytes, which can be injected with, for example, plasmids; plant cell systems, which can be infected with, for example, recombinant virus expression vectors (for example, cauliflower mosaic virus (CaMV) or tobacco mosaic virus (TMV)) or transformed with recombinant plasmid expression vectors containing a nucleic acid sequence encoding the GPCR83 polypeptide or isoform or a functional fragment thereof; or mammalian cell systems (for example, COS, CHO, BHK, HEK293, VERO, Jurkat, HeLa, MDCK, Wi38, and NIH 3T3 cells), which can be transformed with recombinant expression constructs containing, for example, promoters derived, for example, from the genome of mammalian cells (for example, the metallothionein promoter) from mammalian viruses (for example, the adenovirus late promoter and the vaccinia virus 7.5K promoter) or from bacterial cells (for example, the tet-repressor binding its employed in the tet-on and tet-off systems). Also useful as host cells are primary or secondary cells obtained directly from a mammal and transfected with a plasmid vector or infected with a viral vector. Depending on the host cell and the respective vector used to introduce the nucleic acid of the invention the nucleic acid can integrate, for example, into the chromosome or the mitochondrial DNA or can be maintained extrachromosomally like, for example, episomally or can be only transiently comprised in the cells.

In a preferred embodiment, the GPCR83 polypeptide as expressed by such cells is functional and has the expected GPCR83 receptor activity, i.e., upon binding to one or more molecules triggers an activation pathway inside the cell. The same applies to the different isoforms of GPCR83. The cells are preferably mammalian (e.g., human, non-human primate, equine, bovine, sheep, pig, dog, cat, goat, rabbit, mouse, rat, guinea pig, hamster, or gerbil) cells, insect cells, bacterial cells, or fungal (including yeast) cells.

In a further aspect the present invention concerns a pharmaceutical composition, comprising an effective amount of an immunomodulator according to the present invention, a GPCR83 polypeptide or a functional fragment thereof, a polynucleotide encoding a GPCR83 polypeptide or a functional fragment thereof, a vector containing a polynucleotide encoding a GPCR83 polypeptide or a functional fragment thereof, or a host cell recombinantly expressing a GPCR83 polypeptide or a functional fragment thereof, and a pharmaceutically acceptable carrier.

In a preferred embodiment of the pharmaceutical composition according to the present invention, the GPCR83 polypeptide is selected from the group of GPCR83 isoform I polypeptide, GPCR83 isoform 2 polypeptide, GPCR83 isoform 3 polypeptide, and GPCR83 isoform 4 polypeptide or a functional fragment thereof.

In yet another preferred embodiment of the pharmaceutical composition according to the present invention, the GPCR83 polypeptide is the GPCR83 isoform 4 polypeptide

Polypeptides and fragments of the polypeptides useable in the method of the present invention can be modified, for example, for in vivo use by the addition of blocking agents, at the amino- and/or carboxyl-terminal ends, to facilitate survival of the relevant polypeptide in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

The production of pharmaceutical compositions, e.g. in form of medicaments with an effective amount of an immunomodulator according to the present invention, a GPCR83 polypeptide or a functional fragment thereof, a polynucleotide encoding a GPCR83 polypeptide or a functional fragment thereof, a vector containing a polynucleotide encoding a GPCR83 polypeptide or a functional fragment thereof, or a host cell recombinantly expressing a GPCR83 polypeptide or a functional fragment thereof (in the following designated as “active ingredients”) and their uses according to the present invention generally occurs in accordance with standard pharmaceutical technology and methods. For this, the active ingredients, together with pharmaceutical acceptable carriers and/or other suitable pharmaceutical auxiliary agents, are produced into medical forms that are suitable for the different indications, and places of administration.

Thereby, pharmaceutical compositions can be produced having a release rate as desired, e.g. wherein a quick onset and/or a retard- or depot-effect is achieved. Thereby, the pharmaceutical compositions can be an ointment, gel, patch, emulsion, lotion, foam, crème or mixed-phase or amphiphilic emulsion systems (oil/water-water/oil-mix-phase), liposome, transfersome, paste or powder.

According to the present invention, the term “auxiliary agent” shall mean any, non-toxic, solid or liquid filling, diluting or packaging material, as long as it does not adversely react and/or interacts with the active ingredients or the patient. Liquid galenical auxiliary agents, for example, are sterile water, physiological saline, sugar solutions, ethanol and/or oils. Galenical auxiliary agents for the production of tablets and capsules, for example, can contain binders and filling materials.

Furthermore, the active ingredients according to the invention can be used in the form of systemically employed medicaments. These include parenterals belonging to which are injectables and infusions. Injectables are either present in the form of ampoules or as so-called ready-to-use injectables, e.g. as ready-to-use syringes or disposable syringes, and, in addition, are provided in puncture-sealed bottles. The administration of the injectables can take place in form of subcutaneous (s.c.), intramuscular (i.m.), intravenous (i.v.) or intracutaneous (i.c.) application. In particular the suitable forms for injection can be produced as crystal suspensions, solutions, nanoparticular or colloidal-disperse systems, such as, for example, hydrosoles.

The injectable compositions can further be produced as concentrates that are dissolved or dispersed with aqueous isotonic diluents. The infusions can also be prepared in form of isotonic solutions, fatty emulsions, liposome compositions, micro emulsions. Like the injectables, also infusion compositions can be prepared in form of concentrates for dilution. The injectable compositions can also be applied in form of continuous infusions, both in the stationary as well as in the ambulant therapy, e.g. in form of mini pumps.

The active ingredients according to the invention can be bound to a micro carrier or nanoparticle, for example to finely dispersed particles on the basis of poly(meth)acrylates, polylactates, polyglycolates, polyaminoacids or polyetherurethanes. The parenteral compositions can also be modified into a depot preparation, e.g. based on the “multiple unit principle”, if an active ingredient according to the invention is embedded in finely divided or dispersed, suspended form or as crystal suspension, or based on the “single unit principle”, if an active ingredient according to the invention is included in a medicinal form, e.g. in a tablet or a stick that is subsequently implanted. Often, these implants or depot medicaments in the case of “single unit”- and “multiple unit”-medicaments consist of so-called biodegradable polymers, such as, for example polyesters of lactic and glycolic acid, polyether urethanes, polyaminoacids, poly(meth)acrylates or polysaccharides.

As suitable auxiliary agents for producing of parenterals, aqua sterilisata, substances influencing the value of the pH, such as, for example, organic and inorganic acids and bases as well as their salts, buffer substances for adjusting the value of the pH, isotoning agent, such as, for example, sodium chloride, sodium hydrogen carbonate, glucose and fructose, tensides or surface active substances and emulgators, such as, for example, partial fatty acid esters of polyoxyethylene sorbitane (Tween®) or, for example, fatty acid esters of polyoxyethylene (Cremophor®), fatty oils, such as, for example, peanut oil, soy bean oil, and castor oil, synthetic fatty acid esters, such as, for example, ethyloleate, isopropylmyristate and neutral oil (Miglyol®), as well as polymeric auxiliary agents, such as, for example, gelatine, dextran, polyvinylpyrrolidone, solubility enhancing additives, organic solvents, such as, for example, propyleneglycol, ethanol, N,N-dimethylacetamide, propylenglycole or complex-forming substances, such as, for example, citrate and urea, preservatives, such as, for example, benzoic acid hydroxypropylesters and -methylesters, benzylalcohol, antioxidants, such as, for example, sodiumsulfite and stabilisators, such as, for example, EDTA, can be considered.

In suspensions, the addition of thickening agents in order to avoid the setting of the an active ingredient according to the invention, or the addition of tensides, in order to ensure the admixing of the sediment, or of complex forming agents such as, for example, EDTA is possible. Active ingredient complexes can be achieved with different polymers, such as, for example, polyethylene glycoles, polystyrenes, carboxymethyl cellulose, Pluronics® or polyethylene glycolsorbite fatty acid esters. For producing lyophilisates, scaffold forming agents, such as, for example, mannit, dextran, sucrose, human albumin, lactose, PVP or gelatine are used.

The medical forms that are each suitable can be produced in accordance with manuals and procedures known to the person of skill on the basis of pharmaceutical/physical technologies.

A further aspect of the present invention then relates to the respectively produced pharmaceutical composition, comprising an effective amount of an immunomodulator according to the present invention, a GPCR83 polypeptide or a functional fragment thereof, a polynucleotide encoding a GPCR83 polypeptide or a functional fragment thereof, a vector containing a polynucleotide encoding a GPCR83 polypeptide or a functional fragment thereof, or a host cell recombinantly expressing a GPCR83 polypeptide or a functional fragment thereof, and a pharmaceutically acceptable carrier. This pharmaceutical composition can be characterized in that the active ingredient is present in form of a depot substance or as precursor together with a suitable, pharmaceutically acceptable diluent or carrier substance as above.

According to the present invention, the above pharmaceutical composition can be present in the form of tablets, dragees, capsules, droplets, suppositories, compositions for injection or infusion for peroral, rectal or parenteral use. Such administration forms and their production are known to the person of skill.

In a further important aspect the present invention relates to a method of treatment of a human suffering from an undesired immunoreaction, comprising administering to said human an effective amount of a pharmaceutical composition according to the present invention.

An undesired immunoreaction in a human according to the present invention comprises any reaction of the immune system, wherein the homeostasis of the immune system is not maintained. Undesired immunoreactions are for instance any auto-immune diseases such as diabetes type I, rheumatoid arthritis, and Crohn's disease. Further undesired immunoreaction are any forms of allergy or asthma but also any adverse transplant reactions. Further undesired immunoreactions are the undesired suppression of the immune reaction against tumor cells and/or any infections.

Pharmaceutical compositions are generally administered in an amount that is effective for the treatment or prophylaxis of a specific condition or conditions. The initial dose in a human is accompanied by a clinical monitoring of the symptoms, that is, the symptoms of the selected condition.

The suitable and effective dose can be presented as a single dose or as divided doses, in suitable intervals, for example, as two, three, four or more subdoses per day. Suitable dosages can readily be obtained by the person of skill through routine experimentation, and can be based on factors, such as, for example, the concentration of the active drug, the body weight and age of the patient, and other patient- or active drug-related factors.

In another aspect thereof, the present invention relates to a method of treatment of a human suffering from an autoimmune disease, allergy and/or a transplant rejection, comprising the steps of

a) culturing peripheral blood cells of said human comprising conventional T-cells,
b) converting said conventional T-cells in vitro into regulatory T-cells by overexpression of a GPCR83 polypeptide in said conventional T-cells or by contacting said T-cells with an immunomodulator according to the present invention, and
c) re-introducing said converted regulatory T-cells into a mammal, preferably a human.

Methods for converting T-cells are known to the person of skill and can, for example, performed similarly to the expansion of bone marrow cells (CD34+) for transplantation. For an overexpression of GPR83, in addition to retroviral gene transfer, the commercially available nucleofector technology (Amaxa, Germany) could be used.

The converted regulatory T-cells that are re-introduced can be autologous or allogeneic.

In a preferred embodiment of the method according to present invention the GPCR83 polypeptide is selected from the group of GPCR83 isoform 1 polypeptide, GPCR83 isoform 2 polypeptide, GPCR83 isoform 3 polypeptide, and GPCR83 isoform 4 polypeptide or a functional fragment thereof. In a further preferred embodiment of the method according to present invention, the GPCR83 polypeptide is the GPCR83 isoform 4 polypeptide.

If desired, treatment with a modulator of a GPCR of the invention may be combined with any other suitable therapy, preferably immune-related therapy, as is known to the person of skill.

The invention shall now be described further in the following examples with respect to the accompanying drawings, without being limited thereto. For the purposes of the present invention, all references as cited herein are incorporated by reference in their entireties.

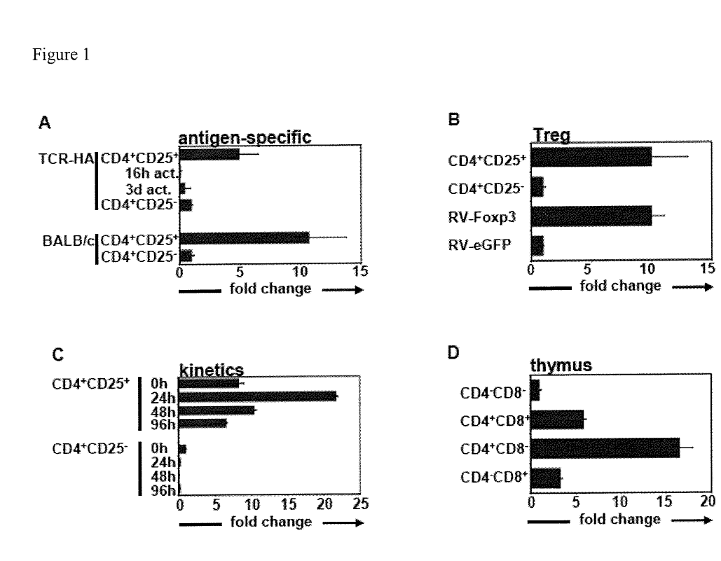

FIG. 1 shows that GPR83 is up-regulated in Tregs of different origin. Total RNA was prepared from sorted freshly isolated, retrovirally infected or in vitro activated T cell populations, as indicated, reverse transcribed, and mRNA expression levels of GPR83 were analyzed by real-time RT-PCR. Relative mRNA amounts were normalized with respect to expression levels in their according naïve, unstimulated or control (RV-eGFP) counterparts (fold change set to 1). RPS9 mRNA expression served as a housekeeping control. Results are from pooled individual mice (n>3). (A) Sorted CD4⁺CD25⁺, CD4⁺CD25⁻ and antigen-stimulated HA-specific T cells were isolated from TCR-HA mice and analyzed for GPR83 expression by real-time RT-PCR in comparison to CD4⁺CD25⁺, CD4⁺CD25⁻ T cells isolated from BALB/c mice. (B) Normalized GPR83 expression in Foxp3 encoding retrovirus or control virus infected naïve T cells as well as sorted CD4⁺CD25⁺ and CD4⁺CD25⁻ T cells isolated from BALB/c mice. (C) CD4⁺CD25⁺ and CD4⁺CD25⁻ T cells were isolated from BALB/c mice and stimulated in vitro with anti-CD3, anti CD28 and IL2 for 24 h, 48 h and 96 h, respectively, prior to GPR83 expression analysis. (D) GPR83 expression in sorted double negative (CD4⁻CD8⁻) (fold change=1), double positive (CD4⁺CD8⁺), and single positive (CD4⁺CD8⁻; CD4⁻CD8⁺) thymocytes isolated from BALB/c mice.

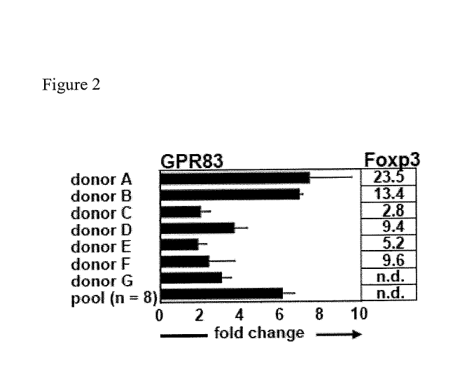

FIG. 2 shows that GPR83 expression in human CD4⁺CD25⁺ Tregs. MACS sorted CD4⁺CD25⁺ and CD4⁺CD25⁻ T cells were isolated from 7 healthy donors and analyzed with a pool of 8 healthy donors for GPR83 and Foxp3 expression by real-time RT-PCR. Relative expression levels in CD4⁺CD25⁺ T cells were normalized to CD4⁺CD25⁻ T cells (fold change=1). RPS9 served as housekeeping control.

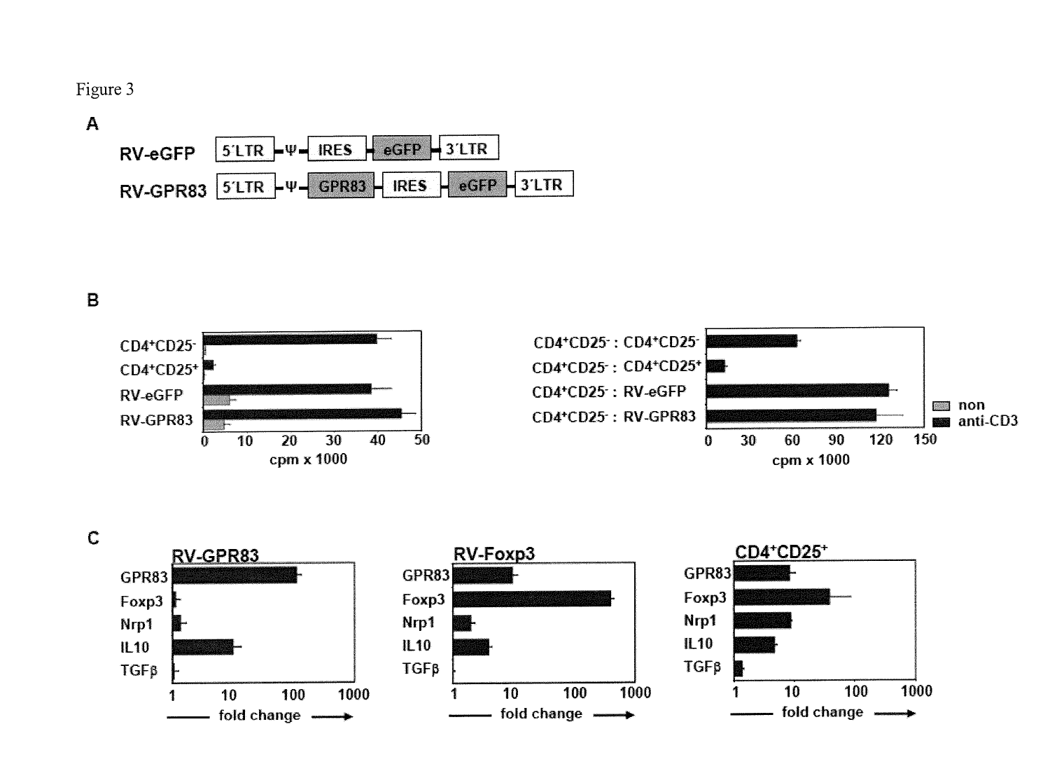

FIG. 3 shows the in vitro analysis of GPR83-transduced CD4⁺CD25⁻ T cells. (A) Schematic drawing of MCSV-based retroviral vector constructs encoding GPR83 and eGFP under control of an internal ribosomal entry side (IRES; RV-GPR83) and the empty control vector (RV-eGFP). (B) Sorted CD4⁺CD25⁺ and CD4⁺CD25⁻ T cells isolated from BALB/c mice or sorted eGFP⁺ T cells one week post infection with retroviral vectors encoding GPR83 and eGFP (RV-GPR83) or the control vector (RV-eGFP) were cultured alone (left) or co-cultured with CD4⁺CD25⁻ T cells (right) in the presence of irradiated APCs with (black bars) or without (grey bars) 1 μg/ml anti-CD3 for 72 h. Proliferation was measured by [³H]-thymidine incorporation; the data represents one of three independent experiment as mean from triplicate wells. (C) Real-time RT-PCR analysis for GPR83, Foxp3, Nrp1, IL10 and TGFβ was performed using reversely transcripted RNA isolated from sorted eGFP⁺ GPR83-transduced (RV-GPR83), Foxp3-transduced naïve T cells one week post infection and freshly isolated CD4⁺CD25⁺ T cells. Expression levels in GPR83-transduced and Foxp3-transduced T cells were normalized for each gene analyzed with respect to expression levels in control virus infected cells (fold change=1), whereas expression in CD4⁺CD25⁺ T cells was normalized to CD4⁺CD25⁻ T cells. RPS9 mRNA expression served as housekeeping gene control. Mean values from at least two independent experiments are shown.

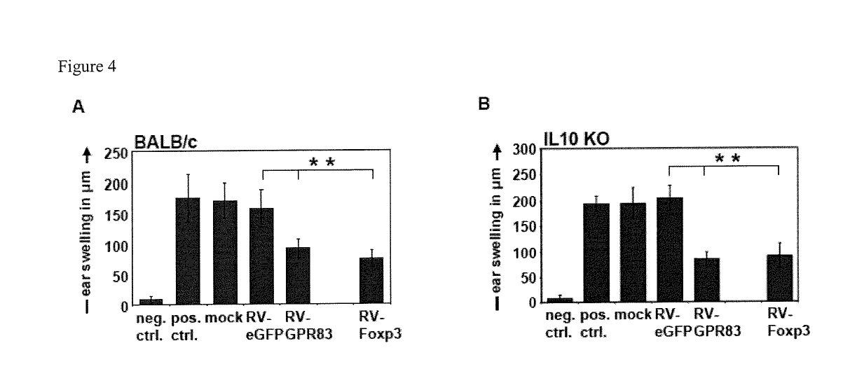

FIG. 4 shows that GPR83-infected CD4⁺CD25⁻ T cells inhibit the effector phase of severe contact hypersensitivity (CHS). Animals were sensitized with DNFB, i.v. injected with 1×10⁶ non-infected (mock), Foxp3-transduced (RV-Foxp3), GPR83-transduced (RV-GPR83) or control virus transduced (RV-eGFP) CD4⁺CD25⁻ T cells isolated from (A) BALB/c or (B) IL10 knock-out mice and ear challenged. As negative control, mice were not sensitized but challenged and as positive control, mice were sensitized and challenged without injecting any cells. Ear swelling was evaluated 36 h after challenge and is expressed as difference between the challenged right ear and the unchallenged left ear. Data are shown as mean±SD of 8 mice in two independent experiments. The Student t test was used to assess the significance of differences.

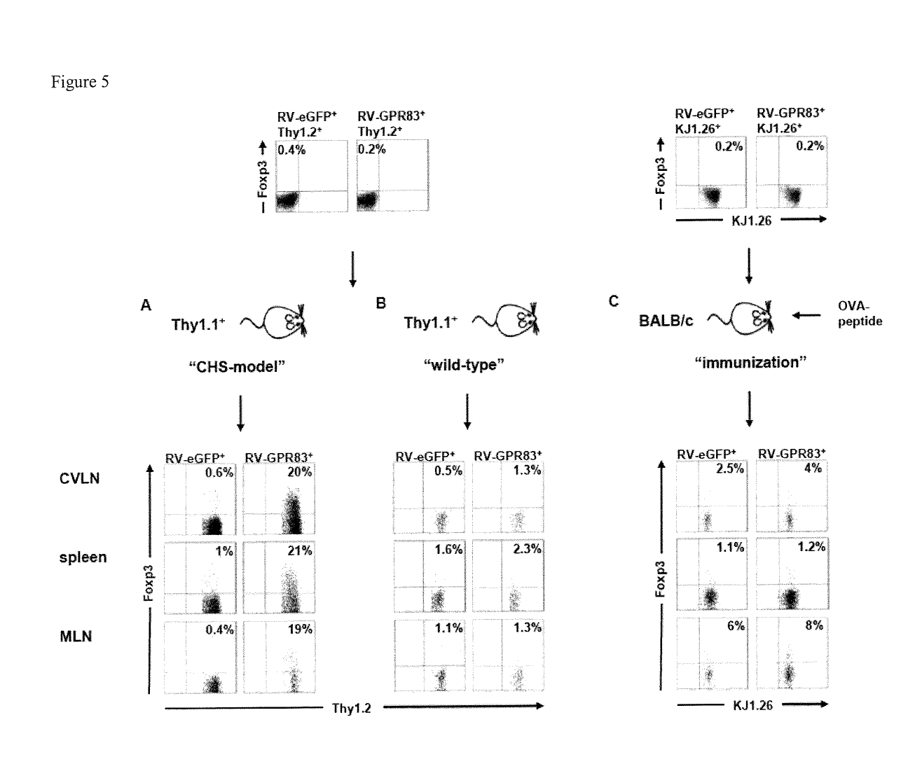

FIG. 5 shows the in vivo suppression during inflammatory immune responses involves Foxp3-conversion in GPR83-transduced T cells. Intracellular Foxp3 stainings of sorted eGFP⁺ control virus (RV-eGFP) or GPR83-transduced (RV-GPR83) CD4⁺ T cells isolated from C57/BL6 Thy1.2⁺ mice (upper panel, left) or KJ1.26⁺ T cells isolated from DO11.10 mice (upper panel, right) one week upon infection. (A) C57/BL6 Thy1.1⁺ mice were sensitized with DNFB, i.v. injected with 7×10⁶ GPR83-transduced (RV-GPR83) or control virus infected (RV-eGFP)C57/BL6 Thy1.2⁺CD4⁺ T cells and ear challenged. 48 h post challenge with DNFB cervical lymph node cells (CVLN), splenocytes (spleen) and mesenteric lymph node (MLN) cells were isolated and analysed for Thy1.2 and Foxp3 expression by flow cytometry. (B) 7×10⁶ GPR83-transduced (RV-GPR83) or control virus infected (RV-eGFP)C57/BL6 Thy1.2⁺CD4⁺ T cells were i.v. injected in healthy C57/BL6 Thy1.1⁺ mice. At day 3 Foxp3 and Thy1.2 expression was analysed by intracellular FACS staining. (C) Wild-type BALB/c mice were immunized with OVA-peptide/CFA one day after transfer of 2.5×10⁶ eGFP⁺KJ1.26⁺ control virus (RV-eGFP) or GPR83-transduced (RV-GPR83) T cells. After 48 h Foxp3 expression was assessed on KJ1.26⁺ T cells re-isolated from cervical lymph nodes (CVLN), spleen and mesenteric lymph nodes (MLN) by flow cytometry.

### EXAMPLES

### Mice

TCR-HA transgenic mice (Kirberg, J., A. Baron, S. Jakob, A. Rolink, K. Karjalainen, and H. von Boehmer 1994. Thymic selection of CD8+ single positive cells with a class II major histocompatibility complex-restricted receptor. J. Exp. Med. 180: 25-34), DO11.10 TCR transgenic mice (Murphy, K. M., A. B. Heimberger, and D. Y. Loh. 1990. Induction by antigen of intrathymic apoptosis of CD4+CD8+TCR1o thymocytes in vivo. Science 250: 1720-1723), BALB/c mice (Harlan, Borchen, Germany), C57/BL6 mice (Harlan, Borchen, Germany) and I110^{tm1Cgn} mice (IL10KO, deficient in IL10; Jackson Laboratories, Bar Harbor, Me.) were housed and bred under specific pathogen-free conditions. B6.PL mice (C57/BL6Thy1.1⁺) were kindly provided from the Bundesinstitut für Risikoforschung, Berlin, Germany. All animal experiments were performed in accordance with institutional, state and federal guidelines.

### Antibodies

The monoclonal antibody 6.5 (anti-TCR-HA) was purified from hybridoma supernatant and used in fluorescein isothiocyanate (FITC)-labeled form. Anti-CD3 (2C11), anti-CD28 (37.51), anti-CD4 (L3T4), anti-CD25 (PC61), anti-CD8 (53-6.7), anti-CD44 (IM7) and anti-Thy1.2 were obtained from BD Biosciences (San Jose, Calif.), anti-DO11.10 TCR (KJ1.26) from Caltag (Burlingame, Calif.) and anti-Foxp3 (FJK-16s) from eBioscience (San Diego, Calif.) and were used unlabeled or as FITC, APC, CyChrome or Phycoerythrin (PE) conjugates.

### Cell Separation and Flow Cytometry

Murine CD4⁺CD25⁻ were enriched from the whole spleen by negative selection using an AutoMACS (Miltenyi Biotec, Bergisch Gladbach, Germany). Human CD4⁺CD25⁺ and CD4⁺CD25⁻ T cells were separated from peripheral blood monocytes (PMBCs) using the regulatory human T cell isolation kit and an AutoMACS separation unit (Miltenyi Biotec, Bergisch Gladbach, Germany) following the manufacturer's instructions. Purity of the enriched cell fractions was >90% as determined by flow cytometry. For gene expression analysis, proliferation and adoptive transfer experiments labeled cells were separated using a MoFlow cell sorter (Cytomation, Fort Collins, Colo.) and purity was >97%. Foxp3 staining was performed using the PE-anti-Foxp3 staining kit from eBioscience according to the manufacturer's recommendations. Flow cytometry analyses were done on a FACScalibur flow cytometer with CellQuest software (BD Biosciences, San Jose, Calif.).

### T Cell Activation

Splenic CD4⁺CD25⁻ T cells or CD4⁺CD25⁺ T cells from BALB/c were FACS-sorted and cultured in the presence of 0.75 μg/ml anti-CD3 (plate bound), 1 μg/ml anti-CD28 (soluble) and 50 U/ml IL2. Different time points after stimulation cells were recovered for RNA preparation. Alternatively, CD4⁺CD25⁻ splenocytes from BALB/c, C57/BL6, IL10KO or DO11.10 mice were stimulated with 0.75 μg/ml anti-CD3 (plate bound) and 1 μg/ml anti-CD28 (soluble) for 48 h prior retroviral infection. For antigen-specific T cell stimulation red blood cell-depleted splenocytes from TCR-HA mice were stimulated with 10 μg/ml hemagglutinin peptide HA₁₁₀₋₁₂₀ for either 16 h or 3 day, respectively. Subsequently, cells were harvested, labelled with anti-CD4, anti-CD25 and 6.5 (anti-TCR-HA), sorted and used for RNA preparation.

### Retroviral Infection

cDNA encoding murine GPR83 or Foxp3 was amplified by RT-PCR from mouse CD4⁺CD25⁺ sorted splenocytes or whole spleen, respectively using specific primers (GPR83: 5′-GGA GCT CAG CCC TTG TGC-3′,5′-TTG TGC CTG TTC TTT TCT GAG C-3′ and Foxp3: 5′-GGA CAA GGA CCC GAT GCC CAA CC-3′ and 5′-CCC TGC CCC CAC CAC CTC TGG-3′), cloned into pCR2.1 TOPO (Invitrogen, Karlsruhe, Germany), sequenced and inserted into a pMCSV-based retroviral vector encoding eGFP under control of an internal ribosomal entry site. These constructs or the empty control vector were used to stably transfect the ecotropic GPE-86⁺ packaging cell line. Concentrated and filtrated (0.45 μm) retrovirus containing culture supernatants supplemented with 20 mM Hepes and 8 μg/ml Polybrene were utilized to infect stimulated CD4⁺CD25⁻ T cells by centrifugation at 500×g for 2 h. Thereafter, cells were transferred to 6-well-plates and incubated at 37° C. and 5% CO₂. After 24 h, half of the culture medium was exchanged and 50 U/ml IL2 added.

### Proliferation Assay

5×10⁴ sorted CD4⁺CD25⁺ and CD4⁺CD25⁻ splenocytes isolated from BALB/c mice and 5×10⁴ GPR83-transduced or control vector infected T cells sorted 1 week post infection were cultured either alone or with 5×10⁴ CD4⁺CD25⁻ T cells isolated from BALB/c mice as responder in the presence of 2.5×10⁵ irradiated BALB/c splenocytes as APCs with 1 μg/ml anti-CD3 for 72 h. Proliferation assays were performed in triplicates in 200 μl of IMDM medium containing 10% fetal calf serum. Cells were pulsed with 1 μCi/well of [³H]-thymidine for the final 8 h or 18 h of the experiment and [³H]-thymidine incorporation was measured by scintillation counting.

### Real-Time RT-PCR

Total RNA was prepared from sorted cell populations using the RNeasy kit (Qiagen, Hilden, Germany) following DNase digestion (Qiagen, Hilden, Germany) and cDNA synthesis by Superscript II Reverse Transcriptase and OligodT mixed with Random Hexamer primers (Invitrogen, Karlsruhe, Germany) according to the manufacturer's recommendations. Real-time RT-PCR was performed in an ABI PRISM cycler (Applied Biosystems) using a SYBR Green PCR kit from Stratagene (La Jolla, Calif.) and specific primers for GPR83 (5′-ACC CTC CCC AGT TCC TTC CTT CAG-3′ and 5′-GGC CAC AAC GGG TTC CAC AGA T-3′), Foxp3; IL10; TGF-0 (Bruder, D., A. M. Westendorf, W. Hansen, S. Prettin, A. D. Gruber, Y. Qian, H. von Boehmer, K. Mahnke, and J. Buer. 2005. On the edge of autoimmunity: T-cell stimulation by steady-state dendritic cells prevents autoimmune diabetes. Diabetes 54: 3395-3401); Nrp1 and RPS9, as described previously (Bruder, D., M. Probst-Kepper, A. M. Westendorf, R. Geffers, S. Beissert, K. Loser, H. von Boehmer, J. Buer, and W. Hansen. 2004. Neuropilin-1: a surface marker of regulatory T cells. Eur. J. Immunol. 34: 623-630).

### Adoptive Transfer of T Cells

Contact hypersensitivity (CHS) experiments with BALB/c, C57/BL6 or B6.PL mice were performed as described elsewhere (Loser, K., W. Hansen, J. Apelt, S. Balkow, J. Buer, and S. Beissert. 2005. In vitro generated regulatory T cells induced by Foxp3-retrovirus infection control murine contact allergy and systemic autoimmunity. Gene Therapy 12: 1294-1304). Briefly, mice were sensitized to DNFB on day 0. On day 4, 1×10⁶ sorted GPR83-, Foxp3-, control virus-transduced or non-transduced CD4⁺CD25⁻ T cells were injected i.v. into each recipient mouse; 24 h prior to elicitation of CHS responses. For immunization 100 μg OVA-peptide/mouse emulsified in Complete Freund's Adjuvant (CFA) were i.p. injected in wild-type mice 24 h after transfer of 2.5×10⁶ sorted OVA-specific control virus or GPR83-infected naïve T cells. Two days later KJ1.26⁺CD4⁺ T cells were analysed for Foxp3 expression.

GPR83 is Highly Expressed by Regulatory T Cells of Different Origin

The inventors initially sought to define a general “Treg-signature”, a set of genes specifically expressed by naturally occurring polyclonal and antigen-specific regulatory T cells. For this purpose they performed extensive gene expression profiling of naturally occurring polyclonal Foxp3⁺CD4⁺CD25⁺ Tregs isolated from BALB/c mice, monoclonal Foxp3⁺CD4⁺CD25⁺ Tregs of known antigen specificity isolated from TCR-HA mice as well as CD4⁺ T cells recently activated with their specific antigen to their naïve Foxp3⁻CD4⁺CD25⁻ T cell counterpart using whole genome Affymetrix MOE430 microarrays. By this approach the inventors identified genes that are co-regulated with Foxp3, i.e. that are highly expressed on monoclonal and polyclonal Tregs without being up-regulated upon T-cell activation (Bruder, D., M. Probst-Kepper, A. M. Westendorf, R. Geffers, S. Beissert, K. Loser, H. von Boehmer, J. Buer, and W. Hansen. 2004. Neuropilin-1: a surface marker of regulatory T cells. Eur. J. Immunol. 34: 623-630). Among these genes associated with Foxp3-dependent transcriptional control in naturally occurring regulatory T cells, the inventors found the G-protein coupled receptor 83 (GPR83) to be co-expressed with Foxp3. These findings are well in line with recently published microarray data of Tregs identified by a fluorescent protein reporter “knocked-in” the Foxp3 locus (Fontenot, J. D., J. P. Rasmussen, L. M. Williams, J. L. Dooley, A. G. Farr, and A. Y. Rudensky. 2005. Regulatory T cell lineage specification by the forkhead transcription factor FoxP3. Immunity 22: 329-341). To investigate the co-regulation of GPR83 and Foxp3 in more detail, the inventors quantified GPR83 mRNA amounts in Foxp3⁺ polyclonal and antigen-specific CD4⁺CD25⁺ Tregs in comparison to their naïve or recently activated CD4⁺CD25⁻ counterparts by real-time RT-PCR. As shown in FIG. 1A, GPR83 was found to be highly up-regulated in naturally occurring Foxp3⁺ Tregs (11-fold) and antigen specific CD4⁺CD25⁺Foxp3⁺ Tregs (5-fold) in contrast to recently activated T cells, that even show a 2 to 10-fold down-regulation of GPR83 mRNA.

The inventors next analyzed whether ectopic expression of Foxp3 in naïve CD4⁺CD25⁻ T cells induces GPR83 expression in these cells. To this end, naïve CD4⁺CD25⁻ T cells were infected with a Foxp3-encoding retrovirus conferring regulatory function to the infected cells (data not shown). Real-time RT-PCR analysis indicated elevated GPR83 levels in Foxp3-transduced T cells that were similar to those observed in the naturally occurring Tregs (FIG. 1B). Extending the inventors' analysis to activated polyclonal CD4⁺CD25⁻ T cells revealed that GPR83 mRNA expression is further down-regulated upon stimulation in vitro. In contrast, naturally occurring CD4⁺CD25⁺ Tregs exhibited a 3-fold increase in GPR83 mRNA content 24 h upon T cell stimulation (FIG. 1C).

Most recently, it was shown that GITR, CTLA4 and Foxp3 expression is initiated at the double positive stage of thymic development; thus, Tregs seem to be positively selected at the CD4⁺CD8⁺ differentiation stage (Cupedo, T., M. Nagasawa, K. Weijer, B. Blom, and H. Spits. 2005. Development and activation of regulatory T cells in the human fetus. Eur. J. Immunol. 35: 383-390, Darrasse-Jeze, G., G. Marodon, B. L. Salomon, M. Catala, and D. Klatzmann. 2005. Ontogeny of CD4+CD25+ regulatory/suppressor T cells in human fetus. Blood 105: 4715-4721). In line with these reports, the inventors could detect increasing GPR83 expression levels along thymic development; elevated GPR83 expression in the double positive compartment (6-fold up-regulation in comparison to the double negative stage) and CD4⁺ single positive stage (17-fold up-regulation) in contrast to double negative and CD8+ single positive thymocytes (FIG. 1D).

In summary, the inventors could clearly demonstrate, that GPR83 is predominantly expressed by naturally occurring polyclonal, antigen-specific and Foxp3-transduced Tregs in contrast to naïve and recently activated ones, thereby exhibiting a similar expression pattern as Foxp3 also during development of Tregs in the thymus.

Considering the GPR83 expression profile in murine regulatory T cells, the question arises if GPR83 is regulated in a similar fashion in human CD4⁺CD25⁺ Tregs. For this purpose, the inventors isolated CD4⁺CD25⁺ and CD4⁺CD25⁻ T cells from peripheral blood of seven healthy donors by MACS sorting and analysed GPR83 and Foxp3 expression by real-time RT-PCR. As shown in FIG. 2 GPR83 is 2-7 fold up-regulated in all individual human CD4⁺CD25⁺ Treg cell populations analysed and also co-regulated with Foxp3 much like it was shown above for murine Tregs (FIG. 1).

### In Vitro Characterisation of GPR83-Transduced Naive T Cells

To better define the biological function of GPR83 expression by Tregs, the inventors constructed MSCV-based retroviral vectors encoding GPR83 and eGFP under control of an internal ribosomal entry side (IRES) (RV-GPR83). In addition, an empty control vector was generated that contained only eGFP (RV-eGFP) (FIG. 3A). Retroviral vectors were stably transfected into GPE86⁺ packaging cells and virus containing supernatants were used to infect naive CD4⁺CD25⁻ T cells.

One week post infection, eGFP⁺ T cells (about 20%) were FACS-sorted, resulting in 99% purity as determined by FACS re-analysis (data not shown). To address whether GPR83-transduced T cells have acquired characteristics of naturally occurring CD4⁺CD25⁺ Tregs, the inventors performed in vitro proliferation assays and investigated the suppressive capacity in co-culture experiments. Naïve T cells infected with RV-eGFP served as controls. As shown in FIG. 3B (left panel), GPR83-transduced T cells exhibited proliferative capacity comparable to freshly isolated CD4+CD25⁻ naïve T cells, whereas CD4⁺CD25⁺ Tregs showed an anergic phenotype. Furthermore, GPR83-transduced T cells in contrast to naturally occurring CD4⁺CD25⁺ Tregs were not able to inhibit proliferation of naïve CD4⁺CD25⁻ T cells in co-culture experiments (FIG. 3B, right panel). Similar results were obtained upon allogenic stimulation in an MLR type assay system. Thus, GPR83-transduction did not confer suppressive capacity in vitro. Moreover, when the inventors analyzed the expression of several genes associated with Treg cell function by quantitative real-time RT-PCR from the retroviral infected cells, the inventors observed that over-expression of GPR83 did not result in an increase of Foxp3, Nrp1 and TGFβ mRNA expression, but induced a 10-fold up-regulation in IL10 mRNA (FIG. 3C). Re-analysis of GPR83-infected T cells upon allogenic stimulation co-cultured with or without congenic naïve T cells in the course of an MLR revealed also no induction in Foxp3 expression.

GPR83-Transduced Naïve T Cells Acquire Suppressive Activity In Vivo

It might be possible that GPR83 itself or GPR83-mediated signals confer suppressive activity to conventional CD4⁺ T cells only under conditions encountered in vivo as mechanisms used by Tregs to interfere with ongoing immune responses are much more complex (von Boehmer, H. 2003. Dynamics of suppressor T cells: in vivo veritas. J. Exp. Med. 198: 845-849.). The inventors therefore examined the capacity of GPR83-transduced T cells to inhibit the effector phase of a contact hypersensitivity (CHS) reaction leading to severe skin inflammation that is T cell-mediated and dependent on dendritic cells (Loser, K., W. Hansen, J. Apelt, S. Balkow, J. Buer, and S. Beissert. 2005. In vitro generated regulatory T cells induced by Foxp3-retrovirus infection control murine contact allergy and systemic autoimmunity. Gene Therapy 12: 1294-1304, Watanabe, H., M. Unger, B. Tuvel, B. Wang, and D. N. Sauder. 2002. Contact hypersensitivity: the mechanism of immune responses and T cell balance. J Interferon Cytokine Res. 22: 407-412).

Groups of naïve BALB/c mice were epicutaneously sensitized to DNFB, i.v. injected with GPR83-transduced (RV-GPR83), Foxp3-transduced (RV-Foxp3) and control virus infected (RV-eGFP) or non infected (mock) T cells and subsequently ear challenged with DNFB. Ear swelling was assessed as a measure of CHS response. As shown in FIG. 4A, mice treated with mock-infected naïve CD4⁺CD25⁻ T cells or control virus infected T cells showed a normal CHS response upon challenge. Interestingly, mice which were adoptively transferred with GPR83-transduced T cells developed a significantly reduced CHS response, which was comparable to the group receiving Foxp3-transduced T cells (RV-Foxp3).

The ability of GPR83-transduced cells to inhibit T cell responses in the CHS reaction could be related to their capacity to make IL10 (FIG. 3C). The inventors therefore studied the influence of this immunosuppressive cytokine on the reduced CHS response observed in the skin after transfer of GPR83-transduced T cells (FIG. 4A). To this end, MACS-sorted CD4⁺CD25⁻ T cells isolated from IL10-deficient (IL10KO) mice were activated in vitro and infected with GPR83-, Foxp3-encoding or control retroviruses. Six days post infection these transduced T cells were analysed for their capacity to inhibit the CHS response. As shown in FIG. 4B IL10-deficient, GPR83-transduced CD4⁺ T cells were able to significantly reduce the CHS response comparable to GPR83-transduced T cells from wild type mice.

Active Suppression In Vivo was Accompanied by the Conversion of GPR83-Transduced, Foxp3⁻ into Foxp3⁺ T Cells

To elucidate the molecular mechanism by which GPR83 infected naïve CD4⁺ T cells acquired their suppressive capacity in vivo, the inventors analysed Foxp3 expression in GPR83-transduced and control virus infected CD4⁺ T cells re-isolated from mice undergoing the CHS response as well as healthy recipients.

For this purpose, C57/BL6 Thy1.1⁺ (B6.PL) mice were sensitized with DNFB, i.v. injected with 7×10⁶ GPR83-transduced (RV-GPR83) or control virus infected (RV-eGFP) CD4⁺Thy1.2⁺ congenic T cells. The inventors could not detect any Foxp3 expression by both infected T cell populations prior to adoptive transfer as determined by FACS analysis shown in FIG. 5 (upper panel, left). Two days after ear challenge with DNFB Foxp3 expression was again quantified by FACS analysis on CD4⁺Thy1.2⁺ T cells re-isolated from the draining lymph nodes as well as the spleen. As depicted in FIG. 5A about 20% of the GPR83-transduced T cells become Foxp3⁺ in the draining lymph node, in contrast to control virus infected T cells. Interestingly, the inventors could also observe an induction of Foxp3 expression to the same extent in GPR83-transduced T cells re-isolated from the spleen and “unaffected” mesenteric lymph nodes (MLN) (FIG. 5A). Therefore, the inventors wondered whether the “in vivo” environment alone is sufficient to induce Foxp3 expression in GPR83-transduced T cells rather than “inflammatory” conditions. However, transfer of 7×10⁶ GPR83- or control virus infected CD4⁺Thy1.2⁺ T cells in congenic Thy1.1⁺ wild-type mice and re-isolation at day 3 did not confer any Foxp3 protein expression in GPR83-transduced T cells as shown in FIG. 5B. To investigate the in vivo induction of Foxp3⁺ Tregs by GPR83 over-expression in more detail, the inventors transferred 2.5×10⁶ OVA-specific control virus or GPR83 infected Foxp3⁻KJ1.26⁺CD4⁺ T cells in wild-type mice prior to immunization with the cognate OVA-peptide in CFA (FIG. 5C, upper panel, right). Re-analysis of the antigen-specific, retroviral infected T cell subsets isolated from cervical (CVLN) and MLN as well as the spleen of immunized mice exhibited no significant up-regulation of Foxp3 in antigen-specific GPR83-transduced T cells (FIG. 5C).

### Measuring GPCR Receptor Activity

One preferred way of measuring GPCR receptor activity is measuring the amount of intracellular calcium upon activation. Even though intracellular calcium levels rise directly only from a G_{q}-protein receptor activation, genetic expression methods have been developed that allow calcium production to proceed upon activation of GPCRs coupled to other G protein types (i.e. Gᵢ/Gₒ or Gₛ). Measuring an intracellular calcium level is commonly known in the art, and is preferably measured by loading the cell with a calcium indicator, such as Oregon Green 488 BAPTA, Fura-2-AM, Fluo-4-AM, and measuring the obtaining fluorescence at a certain emission-wavelength. Further, the amount of released intracellular calcium can be monitored by, for example, the in vitro FLIPR (fluorescence imaging plate readers) assay. In addition, the activity of GPCRs can be also measured by the measurement of one of a variety of other parameters including, for example, IP₃ or cAMP. Additional ways of measuring G-protein coupled receptor activity are known in the art and comprise without limitation electrophysiological methods, transcription assays, which measure, e.g. activation or repression of reporter genes which are coupled to regulatory sequences regulated via the respective G-protein coupled signaling pathway, such reporter proteins comprise, e.g., CAT or LUC; assays measuring internalization of the receptor; or assays in frog melanophore systems, in which pigment movement in melanophores is used as a readout for the activity of adenylate cyclase or phospholipase C (PLC), which in turn are coupled via G-proteins to exogenously expressed receptors (see, for example, McClintock T. S. et al. (1993) Anal. Biochem. 209: 298-305; McClintock T. S, and Lerner M. R. (1997) Brain Res. Brain, Res. Protoc. 2: 59-68, Potenza M N (1992) Pigment Cell Res. 5: 372-328, and Potenza M. N. (1992) Anal. Biochem. 206: 315-322)

### Conversion of T Cells

The level of conversion can be measured by any method suitable to recognize Tregs. Such methods are known to the person skilled in the art. For instance, the level of conversion can be defined by measuring the expression level of the transcription factor FOXP3 (forkhead box p3). The expression of FOXP3 is required for regulatory T cell development and appears to control a genetic program specifying this cell fate. In addition, the two cell surface molecules CD4 and CD25 can be used to define the population of Tregs.

The determination of Foxp3 expression by means of FACS analysis is an accepted method in order to identify Tregs. Furthermore, the suppressive activity can be tested for in vitro. For this, the “potential” Tregs are cultivated with conventional T cells and stimulated. Tregs are able to inhibit the proliferation of conventional T cells (after stimulation). It is a further distinctive feature of both cell types that Tregs, in contrast to conventional T cells, do not proliferate in vitro, and do not produce IL2.

There are a series of molecules that are expressed by Tregs in addition to Foxp3, such as, for example neuropilin1, CTLA4, GITR, and CD103. Nevertheless, these molecules are also expressed by other cells (such as activated T cells), and thus can not be regarded as exclusive marker, such as Foxp3. The most reliable method in order to identify Tregs comprises testing the immunosuppressive function in vivo using one of the many available mouse models.

We claim:

1. A method for identifying a compound capable of interacting with a G-Protein coupled receptor 83 (GPCR83) polypeptide, comprising the steps of
a) contacting the GPCR83 polypeptide or a functional fragment thereof or a host cell recombinantly expressing the GPCR83 polypeptide or a functional fragment thereof with a candidate compound, and
b) determining whether said candidate compound interacts with said GPCR83 polypeptide.

2. The method according to claim 1, wherein said GPCR83 polypeptide is selected from the group of GPCR83 isoform 1 polypeptide, GPCR83 isoform 2 polypeptide, GPCR83 isoform 3 polypeptide, and GPCR83 isoform 4 polypeptide, and functional fragments thereof.

3. The method according to claim 2, wherein said GPCR83 polypeptide is the GPCR83 isoform 4 polypeptide or a functional fragment thereof.

4. The method according to claim 1, wherein said candidate compound is selected from the group of neuropeptides, glucocorticoids, and mast cell products.

5. A compound capable of interacting with a GPCR83 polypeptide, wherein the compound is identified through a method comprising the steps of:
a) contacting the GPCR83 polypeptide or a functional fragment thereof or a host cell recombinantly expressing the GPCR83 polypeptide or a functional fragment thereof with a candidate compound; and
b) determining whether said candidate compound interacts with the GPCR83 polypeptide.

6. A method for identifying a compound capable of functioning as an immunomodulator, comprising the steps of:
a) contacting a host cell recombinantly expressing a GPCR83 polypeptide or a functional fragment thereof with a candidate compound that interacts with a GPCR83 polypeptide; and
b) detecting a response of said host cell compared to a control response as detected in the absence of said candidate compound,
wherein said response indicates that said candidate compound is capable of functioning as an immunomodulator.

7. The method according to claim 6, wherein said GPCR83 polypeptide is selected from the group of GPCR83 isoform 1 polypeptide, GPCR83 isoform 2 polypeptide, GPCR83 isoform 3 polypeptide, and GPCR83 isoform 4 polypeptide, and functional fragments thereof.

8. The method according to claim 7, wherein said GPCR83 polypeptide is the GPCR83 isoform 4 polypeptide or a functional fragment thereof.

9. The method according to claim 6, wherein said candidate compound is selected from the group of neuropeptides, glucocorticoids, and mast cell products.

10. The method according to claim 6, wherein said immunomodulator comprises a compound selected from inducers or suppressors of an immunoreaction.

11. An immunomodulator identified through a method comprising the steps of:
a) contacting a host cell recombinantly expressing a GPCR83 polypeptide or a functional fragment thereof with a candidate compound that interacts with a GPCR83 polypeptide; and
b) detecting a response of said host cell compared to a control response as detected in the absence of said candidate compound,
wherein said response indicates that said candidate compound is capable of functioning as an immunomodulator.

12. A method for identifying a compound capable of functioning as an immunomodulator, comprising the steps of:
a) contacting conventional T-cells with a candidate compound interacting with a GPCR83 polypeptide;
b) detecting the level of conversion of conventional T-cells into regulatory T-cells; and
c) comparing said level of conversion to a control level of conversion as detected in the absence of said candidate compound,
wherein the altered conversion into regulatory T-cells indicates that the candidate compound is capable of functioning as an immunomodulator.

13. The method according to claim 12, wherein said conventional T-cells naturally express a GPCR83 polypeptide, wherein said GPCR polypeptide is selected from the group of GPCR83 isoform 1 polypeptide, GPCR83 isoform 2 polypeptide, GPCR83 isoform 3 polypeptide, and GPCR83 isoform 4 polypeptide.

14. The method according to claim 12, wherein said candidate compound is selected from the group of neuropeptides, glucocorticoids, and mast cell products.

15. The method according to claim 12, wherein said immunomodulator comprises a compound selected from inducers or suppressors of an immunoreaction.

16. An immunomodulator identified through a method comprising the steps of:
a) contacting conventional T-cells with a candidate compound interacting with a GPCR83 polypeptide;
b) detecting the level of conversion of conventional T-cells into regulatory T-cells; and
c) comparing said level of conversion to a control level of conversion as detected in the absence of said candidate compound;
wherein the altered conversion into regulatory T-cells indicates that the candidate compound is capable of functioning as an immunomodulator.

17. A pharmaceutical composition, comprising an effective amount of an immunomodulator, a GPCR83 polypeptide or a functional fragment thereof, a polynucleotide encoding a GPCR83 polypeptide or a functional fragment thereof, a vector containing a polynucleotide encoding a GPCR83 polypeptide or a functional fragment thereof, or a host cell recombinantly expressing a GPCR83 polypeptide or a functional fragment thereof, and a pharmaceutically acceptable carrier;
wherein said immunomodulator is identified by a method selected from:
A) a method comprising the steps of:
a) contacting a host cell recombinantly expressing a GPCR83 polypeptide or a functional fragment thereof with a candidate compound that interacts with a GPCR83 polypeptide; and
b) detecting a response of said host cell compared to a control response as detected in the absence of said candidate compound, wherein said response indicates that said candidate compound is capable of functioning as an immunomodulator; and
B) a method comprising the steps of:
a) contacting conventional T-cells with a candidate compound interacting with a GPCR83 polypeptide;
b) detecting the level of conversion of conventional T-cells into regulatory T-cells; and
c) comparing said level of conversion to a control level of conversion as detected in the absence of said candidate compound;
wherein the altered conversion into regulatory T-cells indicates that the candidate compound is capable of functioning as an immunomodulator.

18. The pharmaceutical composition according to claim 17, wherein said GPCR83 polypeptide is selected from the group of GPCR83 isoform 1 polypeptide, GPCR83 isoform 2 polypeptide, GPCR83 isoform 3 polypeptide, and GPCR83 isoform 4 polypeptide and functional fragments thereof.

19. The pharmaceutical composition according to claim 18, wherein said GPCR83 polypeptide is the GPCR83 isoform 4 polypeptide.

20. A method of treatment of a human suffering from an undesired immunoreaction, comprising administering to said human an effective amount of a pharmaceutical composition comprising a GPCR83 polypeptide or a functional fragment thereof, a polynucleotide encoding a GPCR83 polypeptide or a functional fragment thereof, a vector containing a polynucleotide encoding a GPCR83 polypeptide or a functional fragment thereof, or a host cell recombinantly expressing a GPCR83 polypeptide or a functional fragment thereof, and a pharmaceutically acceptable carrier;
wherein said immunomodulator is identified by a method selected from:
A) a method comprising the steps of:
a) contacting a host cell recombinantly expressing a GPCR83 polypeptide or a functional fragment thereof with a candidate compound that interacts with a GPCR83 polypeptide; and
b) detecting a response of said host cell compared to a control response as detected in the absence of said candidate compound,
wherein said response indicates that said candidate compound is capable of functioning as an immunomodulator; and
B) a method comprising the steps of:
a) contacting conventional T-cells with a candidate compound interacting with a GPCR83 polypeptide;
b) detecting the level of conversion of conventional T-cells into regulatory T-cells; and
c) comparing said level of conversion to a control level of conversion as detected in the absence of said candidate compound;
wherein the altered conversion into regulatory T-cells indicates that the candidate compound is capable of functioning as an immunomodulator.

21. A method of treatment of a human suffering from an autoimmune disease, allergy and/or a transplant rejection, comprising the steps of
a) culturing peripheral blood cells of said human comprising conventional T-cells;
b) converting said conventional T-cells in vitro into regulatory T-cells by overexpression of a GPCR83 polypeptide in said conventional T-cells or by contacting said T-cells with an immunomodulator; and
c) re-introducing said converted regulatory T-cells into a human;
wherein said immunomodulator is identified by a method selected from:
A) a method comprising the steps of:
a) contacting a host cell recombinantly expressing a GPCR83 polypeptide or a functional fragment thereof with a candidate compound that interacts with a GPCR83 polypeptide; and
b) detecting a response of said host cell compared to a control response as detected in the absence of said candidate compound,
wherein said response indicates that said candidate compound is capable of functioning as an immunomodulator; and
B) a method comprising the steps of:
a) contacting conventional T-cells with a candidate compound interacting with a GPCR83 polypeptide;
b) detecting the level of conversion of conventional T-cells into regulatory T-cells; and
c) comparing said level of conversion to a control level of conversion as detected in the absence of said candidate compound;
wherein the altered conversion into regulatory T-cells indicates that the candidate compound is capable of functioning as an immunomodulator.

22. The method according to claim 21, wherein said GPCR83 polypeptide is selected from the group of GPCR83 isoform 1 polypeptide, GPCR83 isoform 2 polypeptide, GPCR83 isoform 3 polypeptide, and GPCR83 isoform 4 polypeptide and functional fragments thereof.

23. The method according to claim 22, wherein said GPCR83 polypeptide is the GPCR83 isoform 4 polypeptide.
